(19) United States
(12) Patent Application Publication
(10) Pub. No.: US 2017/0042503 A1
(43) Pub. Date: Feb. 16, 2017

(54) RESPIRATORY FUNCTION TESTING SYSTEM AND RESPIRATORY FUNCTION TESTING METHOD THEREOF

(71) Applicants: CHIA-CHI SU, Taichung (TW); HSIAO-PAO YEN, New Taipei (TW); PEI-LING HSU, New Taipei (TW); CHIA-HUNG CHEN, New Taipei (TW)

(72) Inventors: CHIA-CHI SU, Taichung (TW); HSIAO-PAO YEN, New Taipei (TW); PEI-LING HSU, New Taipei (TW); CHIA-HUNG CHEN, New Taipei (TW)

(21) Appl. No.: 15/232,809

(22) Filed: Aug. 10, 2016

Related U.S. Application Data
(60) Provisional application No. 62/203,951, filed on Aug. 12, 2015.

(51) Int. Cl. A61B 7/00; A61B 7/04; A61B 8/08; A61B 5/087; A61B 5/091

(52) U.S. Cl. CPC A61B 7/003; A61B 5/0871; A61B 5/087; A61B 5/091; A61B 8/08; A61B 7/04

(57) ABSTRACT

A respiratory function testing system includes an air transforming device, a sound reception device and an operation device. The air transforming device is configured to collect exhaled air for a predetermined period and generate a full-range sound signal according to the collected exhaled air. The full-range sound signal at least contains an ultrasonic signal. The sound reception device is configured to receive and record the full-range sound signal. The operation device is in communication with the sound reception device and is configured to receive and compute the ultrasonic signal contained in the full-range sound signal recorded by the sound reception device to generate a respiratory function parameter. A respiratory function testing method of the respiratory function testing system is also provided.

### FIELD OF THE INVENTION

The present invention relates to respiratory function testing system and respiratory function testing method thereof, and more particularly to respiratory function testing system and respiratory function testing method thereof utilizing ultrasonic signals generated by exhaled air of a user.

### BACKGROUND OF THE INVENTION

Current common spirometry on the market is mainly plastic pressure indicator based or turbine based. For a spirometry with plastic pressure indicator based, the pressure generated by the exhalation blowing into the spirometry is for driving the sensor/receptor disposed at the end or side of the spirometry to generate a corresponding expiratory signal. This type of spirometry has an uncomplicated structure; however, it is impossible to continuously monitor the expiratory signal within one expiratory period. For a spirometry with turbine based, the pressure generated by the exhalation blowing into the spirometry is for driving the fan disposed in the spirometry to rotate. Through measuring the current generated by the rotating fans or using infrared technology, the cycles or speed of the rotations of the fans is counted; and therefore, data related to respiratory functions within one expiratory period is calculated based on the number or speed of the rotations of the fans.

### SUMMARY OF THE INVENTION

One objective of the present invention is to provide a respiratory function testing system, wherein the respiratory function testing technical adopted by the respiratory function testing system is different from the spirometry mentioned in BACKGROUND OF THE INVENTION.

Another objective of the present invention is to provide a respiratory function testing method applicable to the respiratory function testing system.

The present invention provides a respiratory function testing system, which includes an air transforming device, a sound reception device and an operation device. The air transforming device is configured to collect exhaled air for a predetermined period and generate a full-range sound signal according to the collected exhaled air. The full-range sound signal at least contains an ultrasonic signal. The sound reception device is configured to receive and record the full-range sound signal. The operation device is in communication with the sound reception device and is configured to receive and compute the ultrasonic signal in the full-range sound signal recorded by the sound reception device to calculate respiratory function parameters.

The present invention provides a respiratory function testing method applicable to the above respiratory function testing system. The respiratory function testing method includes: collecting exhaled air for a predetermined period and generating a full-range sound signal according to the collected exhaled air, wherein the full-range sound signal at least contains an ultrasonic signal; receiving and recording the full-range sound signal; and computing the ultrasonic signal in the full-range sound signal to generate corresponding respiratory function parameters.

In summary, by sequentially configuring the air transforming device to collect exhaled air for a predetermined period and generate a full-range sound signal according to the collected exhaled air, configuring the sound reception device to receive and record the full-range sound signal and configuring the operation device to receive and compute the ultrasonic signal contained in the full-range sound signal to generate a respiratory function parameter, the respiratory function testing system and the respiratory function testing method of the present invention can determine whether a user has a normal respiratory function.

### BRIEF DESCRIPTION OF THE DRAWINGS

Other advantages, objectives and features of the present invention will become apparent from the following description referring to the attached drawings.

FIG. 1 is a schematic diagram of a respiratory function testing system in accordance with an embodiment of the present invention;

FIG. 2 is a schematic plot of full-range sound signal versus time in accordance with an embodiment of the present invention;

FIG. 3 is a schematic plot of sound pressure-time curve in accordance with an embodiment of the present invention;

FIG. 4 is a schematic plot of flow-time curve in accordance with an embodiment of the present invention;

FIG. 5 is a schematic plot of volume-time curve in accordance with an embodiment of the present invention; and

FIG. 6 is a flow chart of a respiratory function testing method in accordance with an embodiment of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

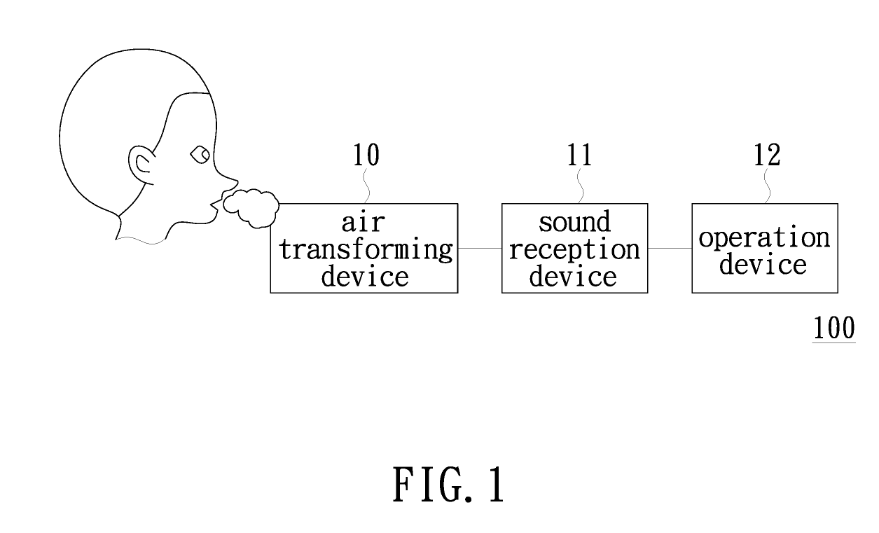

FIG. 1 is a schematic diagram of a respiratory function testing system in accordance with an embodiment of the present invention. As shown in FIG. 1, the respiratory function testing system 100 of the present embodiment includes an air transforming device 10, a sound reception device 11 and an operation device 12. The air transforming device 10 is configured to collect exhaled air for a predetermined period and generate a full-range sound signal according to the collected exhaled air, wherein the full-range sound signal at least contains an ultrasonic signal. In the present embodiment, the aforementioned predetermined period is, for example, the duration of a user continuously exhaling air toward the air transforming device 10. The sound reception device 11 is configured to receive and record the full-range sound signal generated by the air transforming device 10. In the present embodiment, the full-range sound signal generated by the air transforming device 10 according to the exhalation of the user covers all the frequency bands higher than 20 KHz; and specifically, the sound reception device 11 is configured to continuously receive and record the full-range sound signal at a frequency higher than 20 KHz for the predetermined period. Although different users may have different durations of exhalation due to the different respective respiratory functions, the respiratory function testing system 100 of the present invention is capable of testing the respiratory functions for the predetermined period for different users.

In the present embodiment, the sound reception device 11 is a microelectromechanical system (MEMS) or a microphone. Specifically, the sound reception device 11 is a highly-sensitive microphone capable of receiving and recording full-range sound signals and is selected from a group consisting of: omnidirectional microphone, cardioid microphone, hypercardioid microphone, shotgun microphone and bi-directional microphone. Because of having sensitive sound reception functions, each one of the microphones in the aforementioned group can be used to receive and record full-range sound signals and store the recorded full-range sound signal as an audio file, wherein the audio length of the audio file is the aforementioned predetermined period. The operation device 12 has a communicating connection with the sound reception device 11. The operation device 12 is configured to receive and compute the ultrasonic signal contained in the full-range sound signal recorded by the sound reception device 11 to generate a respiratory function parameter. In the present embodiment, the aforementioned communicating connection between the sound reception device 11 and the operation device 12 may be implemented via Bluetooth or Wi-Fi (wireless) means, though which the operation device 12 can receive the audio file stored by the sound reception device 11. In the present embodiment, the operation device 12 is an electronic device having computing capability such as smart phone or tablet, and the present invention is not limited thereto.

In one embodiment, the air transforming device 10 includes one or more silent whistles or Galton's whistles (not shown). The silent whistle or Galton's whistle is configured to generate the ultrasound signal according to the exhaled air while the user exhales air toward the air transforming device 10. The air transforming device 10 may include other types of ultrasound generator devices as long as such device is capable of generating the ultrasound signal according to the exhaled air of the user, and the present invention is not limited thereto.

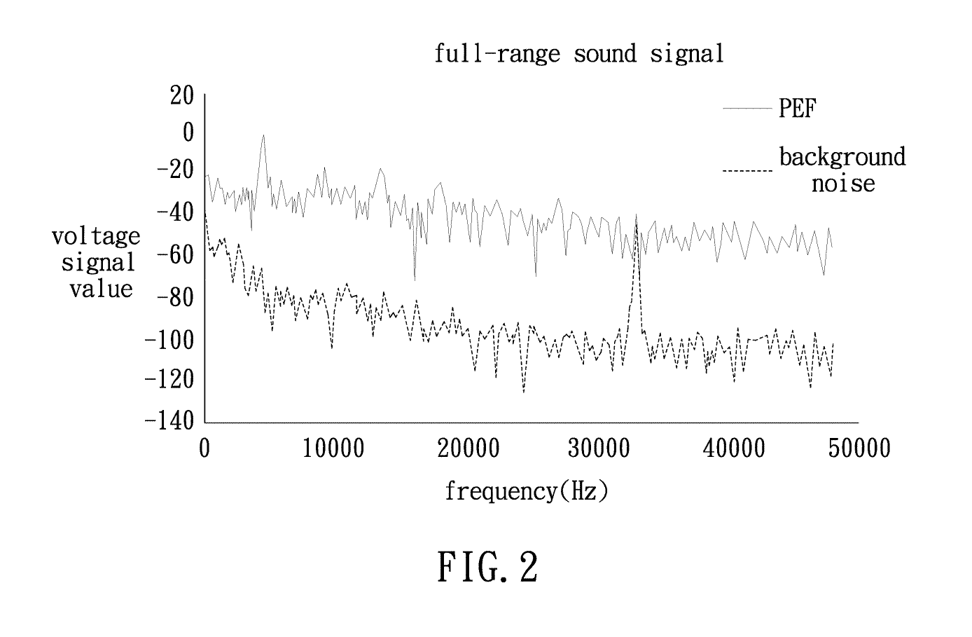

FIG. 2 is a schematic plot of full-range sound signal versus time in accordance with an embodiment of the present invention, wherein the full-range sound signal is represented by voltage signal values. After the operation device 12 receives the audio file of the full-range sound signal (FIG. 2) from the sound reception device 11, the user can select the full-range sound signal at a specific frequency for computing via an application program installed in the operation device 12. The purpose of the frequency selection is for reducing the interference from background noise in the environment and thereby increasing the accuracy of the computation. Specifically, the operation device 12 is configured to capture the sound pressure corresponding to the full-range sound signal at a predetermined frequency. The predetermined frequency may be determined by the application program automatically or set by the user. The aforementioned sound pressure is referred as the volume of the full-range sound signal and measured in decibels (dB) or fast Fourier transform (FFT). Full-range sound signal may have different sound pressures at different frequencies; therefore, by a proper frequency selection, a qualifying computing result may still be obtained if the user has a smaller amount of exhalation.

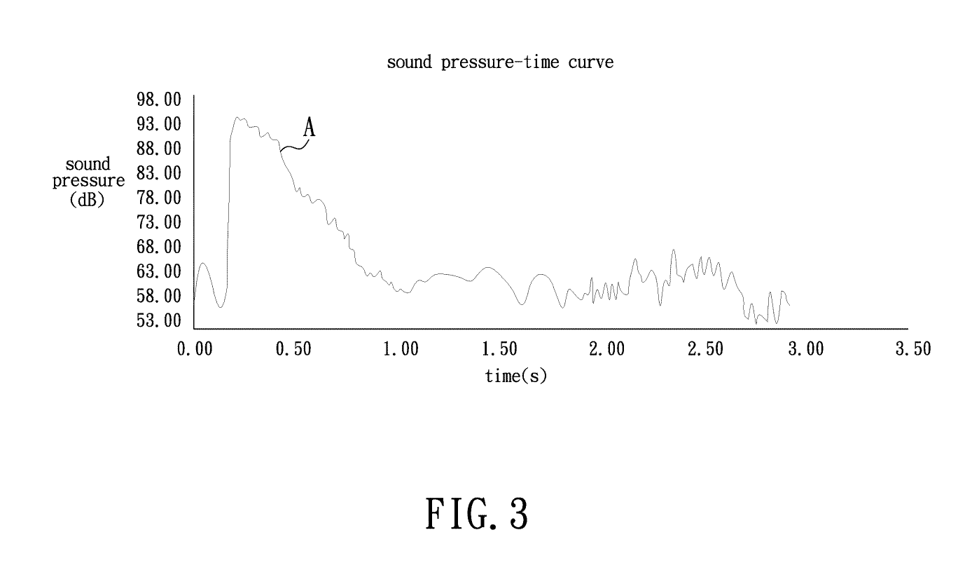

FIG. 3 is a schematic plot of sound pressure-time curve in accordance with an embodiment of the present invention. In FIG. 3, the curve A represents the sound pressures corresponding to the exhalation of the user within 0-3 seconds once a specific frequency is selected by the operation device 12; wherein the horizontal axis represents the dimension of time (e.g., in seconds) and the vertical axis represents the dimension of sound pressure (e.g., in dB). Specifically, the operation device 12 is configured to perform a transforming operation on the audio file of the ultrasonic signal contained in the full-range sound signal recorded by the sound reception device 11 to generate the respiratory function parameters. In the present embodiment, the respiratory function parameter includes peak expiratory flow (PEF), forced expiratory volume 1 (FEV1) and forced vital capacity (FVC).

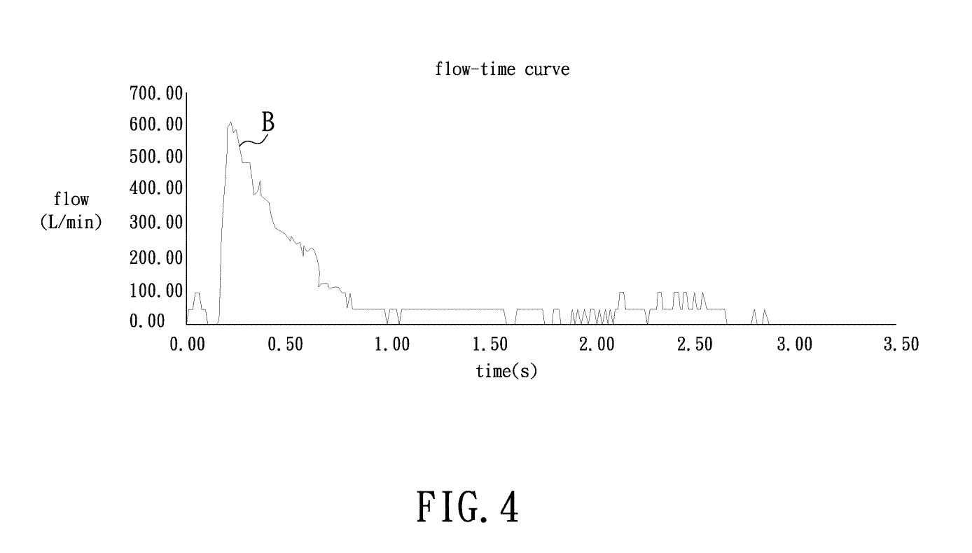

FIG. 4 is a schematic flow-time curve in accordance with an embodiment of the present invention, wherein the plot is transformed from FIG. 3 through a regression equation (e.g., a polynomial of one or more than one degrees such as y=ax+b, y=ax²+bx+c). Specifically, the curve B in FIG. 4 is obtained by comparing the curve A in FIG. 3 with the regression equation and verified with the spirometry certified by the US Food and Drug Administration (FDA). In the regression equation, y stands a testing value derived from a spirometry certified by FDA and x stands a testing value derived from the respiratory function testing system 100 of the present embodiment. In FIG. 4, PEF is referred as the maximum value of the curve B within a predetermined period; specifically, the PEF in FIG. 4 is 614.78 L/min and occurs at the point 0.22 in time.

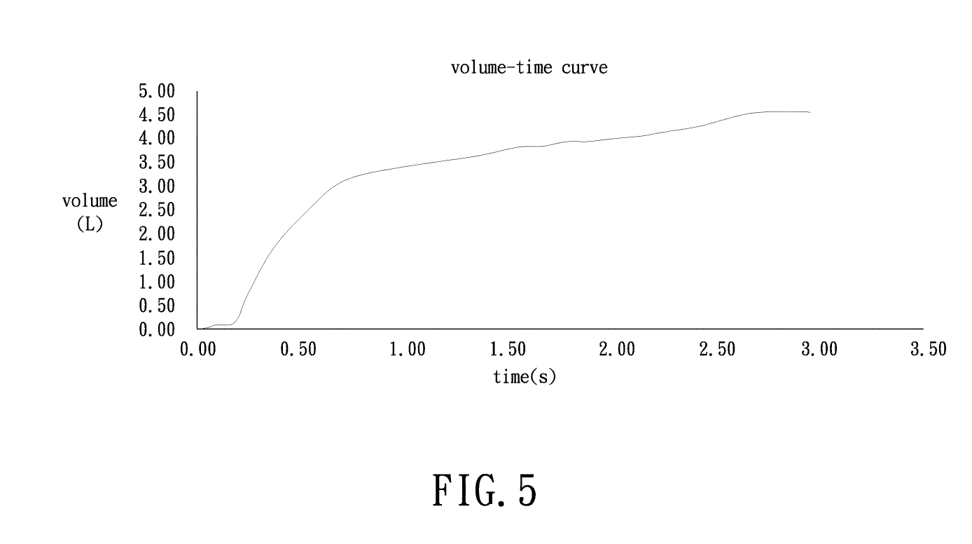

FIG. 5 is a schematic volume-time curve in accordance with an embodiment of the present invention, wherein the plot is transformed from FIG. 4. Specifically, the curve in FIG. 5 is obtained by performing the trapezoidal area integration formula on the curve B in FIG. 4 to accumulate the areas covered by the curve B. FEV1 is referred as the volume of expiration in the first second. In FIG. 5, for example, FEV1 is the value of volume corresponding to the point 1 in time. Further, FVC is referred as the volume of expiration within 0-3 seconds; that is, FVC is referred as the volume of expiration within a predetermined period of one respiratory function test. In FIG. 5, for example, FVC is the value of volume corresponding to the point 3 in time.

After all of the PEF, FEV1 and FVC are calculated, the condition of the respiratory functions of the user can be determined through comparing the calculated PEF, FEV1 and FVC with respective determined standard values. In general, the standard value of PEF is higher than 80% and the standard value of ratio of FVC to FEV1 (FVC/FEV1) is higher than 70%. Therefore, for an asthma patient, it is determined that the patient has a proper treatment if the variation (%) of PEF is lower than 20%; it is determined that the patient may need to increase the amount of medicine if the variation of PEF is in a range between 20%-30%; and it is determined that the patient is having asthma and may need an emergency treatment if the variation of PEF is higher than 30%. Herein the variation (%) of PEF is referred as: ((the maximum PEF)−(the minimum PEF))/((the maximum PEF)+(the minimum PEF))*100%.

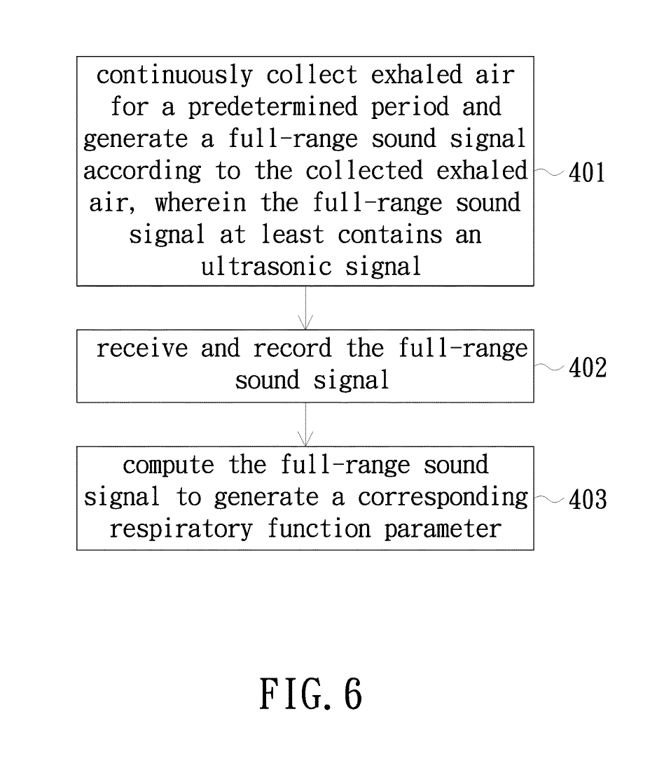

FIG. 6 is a flow chart of a respiratory function testing method in accordance with an embodiment of the present invention. As shown in FIG. 6, the respiratory function testing method of the present embodiment is applicable to the respiratory function testing system 100 and includes steps 401-403. Specifically, step 401: continuously collecting exhaled air for a predetermined period and generating a full-range sound signal according to the collected exhaled air, wherein the full-range sound signal at least contains an ultrasonic signal. Step 402: receiving and recording the full-range sound signal. Step 403: computing the full-range sound signal to generate a corresponding respiratory function parameter.

Refer to Table 1, which is a comparison between the PEF derived from the respiratory function testing system of the present invention and the PEF derived from the spirometry certified by FDA (hereunder is referred as a comparative example). As shown in Table 1, there are thirteen participants involved to the comparison; specifically, each one of the participants repeats the spirometric experiments three times for both of the systems of the present invention and the comparative example. The results of experiments indicate that all of the error rates of the system of the present invention relative to the comparative example are lower than 7%. Therefore, it is shown that the accuracy of the respiratory function testing system of the present invention is as good as that of the spirometry certified by FDA.

TABLE 1
Comparison of PEF between the Present Invention
and Comparative Examples

| | | | | Comparative | Present | |
|---|---|---|---|---|---|---|
| | | | | Examples | Invention | |
| | | | Height | PEF | PEF | Error |
| Participants | Gender | Age | (cm) | (L/min) | (L/min) | Rates |
| 1 | M | 23 | 167 | 619 | 657.75 | 6% |
| 2 | F | 10 | 140 | 286 | 277.68 | 3% |
| 3 | F | 30 | 159 | 341 | 346.88 | 2% |
| 4 | F | 28 | 153 | 375 | 355.99 | 5% |
| 5 | F | 28 | 153 | 356 | 344.20 | 3% |
| 6 | M | 40 | 176 | 618 | 614.91 | 1% |
| 7 | F | 46 | 156 | 367 | 364.68 | 1% |
| 8 | F | 28 | 153 | 366 | 356.29 | 3% |
| 9 | F | 28 | 153 | 380 | 361.81 | 5% |
| 10 | M | 40 | 176 | 622 | 615.76 | 1% |
| 11 | M | 58 | 168 | 545 | 554.91 | 2% |
| 12 | M | 40 | 176 | 588 | 629.67 | 7% |
| 13 | F | 28 | 153 | 365 | 374.77 | 3% |

In summary, by sequentially configuring the air transforming device to collect exhaled air for a predetermined period and generate a full-range sound signal according to the collected exhaled air, configuring the sound reception device to receive and record the full-range sound signal and configuring the operation device to receive and compute the ultrasonic signal contained in the full-range sound signal to generate a respiratory function parameter, the respiratory function testing system and the respiratory function testing method of the present invention can determine whether a user has a normal respiratory function.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A respiratory function testing system, comprising:
an air transforming device, configured to collect exhaled air for a predetermined period and generate a full-range sound signal according to the collected exhaled air, wherein the full-range sound signal comprises an ultrasonic signal;
a sound reception device, configured to receive and record the full-range sound signal; and
an operation device, communicating with the sound reception device and configured to receive and compute the ultrasonic signal in the full-range sound signal recorded by the sound reception device to generate a respiratory function parameter.

2. The respiratory function testing system according to claim 1, wherein the air transforming device comprises a silent whistle or a Galton's whistle.

3. The respiratory function testing system according to claim 1, wherein the sound reception device comprises a microelectromechanical system or a microphone, and the microphone is selected from a group consisting of: an omnidirectional microphone, a cardioid microphone, a hypercardioid microphone, a shotgun microphone and a bi-directional microphone.

4. The respiratory function testing system according to claim 1, wherein the operation device is further configured to capture a sound pressure corresponding to the full-range sound signal at a predetermined frequency and the sound pressure corresponding to the predetermined frequency is adapted for reducing interference from a background noise.

5. The respiratory function testing system according to claim 4, wherein the respiratory function parameter comprises peak expiratory flow (PEF), forced expiratory volume 1 (FEV1) and forced vital capacity (FVC), and the operation device is further configured to compute the captured sound pressure to generate the PEF, the FEV1 and the FVC.

6. A respiratory function testing method of a respiratory function testing system, the respiratory function testing system comprising an air transforming device, a sound reception device and an operation device, and the respiratory function testing method comprising:
collecting exhaled air for a predetermined period and generating a full-range sound signal according to the collected exhaled air, wherein the full-range sound signal comprises an ultrasonic signal;
receiving and recording the full-range sound signal; and
computing the ultrasonic signal in the full-range sound signal to generate a corresponding respiratory function parameter.

7. The respiratory function testing method according to claim 6, wherein the air transforming device comprises a silent whistle or a Galton's whistle, and the silent whistle or the Galton's whistle is configured to generate the full-range sound signal according to the collected exhaled air.

8. The respiratory function testing method according to claim 6, wherein the sound reception device comprises a microelectromechanical system or a microphone and the microphone is configured to receive and record the full-range sound signal.

9. The respiratory function testing method according to claim 6, further comprising:
capturing a sound pressure corresponding to the full-range sound signal at a predetermined frequency.

10. The respiratory function testing method according to claim 9, wherein the respiratory function parameter comprises peak expiratory flow (PEF), forced expiratory volume 1 (FEV1) and forced vital capacity (FVC), and the operation device is configured to capture and compute the captured sound pressure to generate the PEF, the FEV1 and the FVC.
